SEQUENCE LISTING (38 sequences)

SEQ ID NO: 1 - DNA, 42 bp, Artificial Sequence
  Synthesized oligonucleotide for primer
aatcgggatccgcggccgcagaggcggtttgcgtattggagc

SEQ ID NO: 2 - DNA, 51 bp, Artificial Sequence
  Synthesized oligonucleotide for primer
ggatttgactacgggcctaaagtcgacagaataaataaatcctggtgtccc

SEQ ID NO: 3 - DNA, 42 bp, Artificial Sequence
  Synthesized oligonucleotide for primer
acggagcatgcgcggccgctcaaaatcggtggagctgcatga

SEQ ID NO: 4 - DNA, 54 bp, Artificial Sequence
  Synthesized oligonucleotide for primer
gggacaccaggatttatttattctgtcgactttaggcccgtagtctgcaaatcc

SEQ ID NO: 5 - DNA, 21 bp, Artificial Sequence
  Synthesized oligonucleotide for primer
cacttattcaggcgtagcacc

SEQ ID NO: 6 - DNA, 24 bp, Artificial Sequence
  Synthesized oligonucleotide for primer
atcggcattttctttgcgttttta

SEQ ID NO: 7 - DNA, 22 bp, Artificial Sequence
  Synthesized oligonucleotide for primer
gaagtaccgagagtctttatcc

SEQ ID NO: 8 - DNA, 38 bp, Artificial Sequence
  Synthesized oligonucleotide for primer
ctacgcctgaataagtggtactgtttctcctcacaacg

SEQ ID NO: 9 - DNA, 40 bp, Artificial Sequence
  Synthesized oligonucleotide for primer
gcaaagaaaatgccgatccgtcaagcaaactgttgaaatc

SEQ ID NO: 10 - DNA, 22 bp, Artificial Sequence
  Synthesized oligonucleotide for primer
tcgtttccataaccgctcttgc

SEQ ID NO: 11 - DNA, 39 bp, Artificial Sequence
  Synthesized oligonucleotide for primer
caacagtttgcttgacgggtactgtttctcctcacaacg

SEQ ID NO: 12 - DNA, 40 bp, Artificial Sequence
  Synthesized oligonucleotide for primer
gtgaggagaaacagtacccgtcaagcaaactgttgaaatc

SEQ ID NO: 13 - DNA, 24 bp, Artificial Sequence
  Synthesized oligonucleotide for primer
gaaattaatcgcggcggaaaaccc

SEQ ID NO: 14 - DNA, 24 bp, Artificial Sequence
  Synthesized oligonucleotide for primer
gtatagatgagtataaaatgtatc

SEQ ID NO: 15 - DNA, 39 bp, Artificial Sequence
  Synthesized oligonucleotide for primer
ctacgcctgaataagtggttgattagaatgactccgcac

SEQ ID NO: 16 - DNA, 37 bp, Artificial Sequence
  Synthesized oligonucleotide for primer
gcaaagaaaatgccgatatgattgttgcaaatatgtc

SEQ ID NO: 17 - DNA, 21 bp, Artificial Sequence
  Synthesized oligonucleotide for primer
ggtggcgttatgtgataatac

SEQ ID NO: 18 - DNA, 38 bp, Artificial Sequence
  Synthesized oligonucleotide for primer
caccacaaaagcggttgttgattagaatgactccgcac

SEQ ID NO: 19 - DNA, 36 bp, Artificial Sequence
  Synthesized oligonucleotide for primer
tggaaggagtaacactatgattgttgcaaatatgtc

SEQ ID NO: 20 - DNA, 20 bp, Artificial Sequence
  Synthesized oligonucleotide for primer
aaccgcttttgtggtgacca

SEQ ID NO: 21 - DNA, 21 bp, Artificial Sequence
  Synthesized oligonucleotide for primer
agtgttactccttccataaag

SEQ ID NO: 22 - DNA, 22 bp, Artificial Sequence
  Synthesized oligonucleotide for primer
gaaagagttttgtgatgtggcg

SEQ ID NO: 23 - DNA, 44 bp, Artificial Sequence
  Synthesized oligonucleotide for primer
ctttatggaaggagtaacactatgattggcatttactcgcctgg

SEQ ID NO: 24 - DNA, 44 bp, Artificial Sequence
  Synthesized oligonucleotide for primer
gtaaaacgacggccagtgccttaataataaaccgcattaacctg

SEQ ID NO: 25 - DNA, 26 bp, Artificial Sequence
  Synthesized oligonucleotide for primer
ggcactggccgtcgttttacaacgtc

SEQ ID NO: 26 - DNA, 23 bp, Artificial Sequence
  Synthesized oligonucleotide for primer
gcgctttctcatagctcacgctg

SEQ ID NO: 27 - DNA, 23 bp, Artificial Sequence
  Synthesized oligonucleotide for primer
caccgaactgagatacctacagc

SEQ ID NO: 28 - DNA, 43 bp, Artificial Sequence
  Synthesized oligonucleotide for primer
ctttatggaaggagtaacactatgcaaggtaatgcactaaccg

SEQ ID NO: 29 - DNA, 44 bp, Artificial Sequence
  Synthesized oligonucleotide for primer
caactttatggaaggagtaacactatgtctgaaagacatttacc

SEQ ID NO: 30 - DNA, 23 bp, Artificial Sequence
  Synthesized oligonucleotide for primer
gttgcgcgtcatgatggctttcc

SEQ ID NO: 31 - DNA, 25 bp, Artificial Sequence
  Synthesized oligonucleotide for primer
gaagtttgggtggcaacagacgatc

SEQ ID NO: 32 - DNA, 25 bp, Artificial Sequence
  Synthesized oligonucleotide for primer
cagggttttcccagtcacgacgttg

SEQ ID NO: 33 - DNA, 1170 bp, Escherichia coli
atgcaaggtaatgcactaaccgttttattatccggtaaaaaatatctgctattgcaggggccaatgggaccctttttcagtgatgttgccgagtggctagagtcattaggtcgtaacgctgtgaatgttgtattcaacggtggggatcgtttttactgccgccatcgacaatacctagcttactaccagacaccgaaagagtttcccggatggttacgggatctccaccggcaatatgactttgacacaatcctctgctttggcgactgccgcccattgcataaagaagcaaaacgctgggcaaagtcgaaagggatccgcttcctggcatttgaagaaggatatttacgcccgcaatttattaccgttgaagaaggcggagtgaacgcatattcatcgctaccgcgcgatccggatttttatcgtaagttaccagatatgcctacgccgcacgttgagaacttaaaaccttcaacgatgaaacgtataggccatgctatgtggtattacctgatgggctggcattaccgtcatgagtttcctcgctaccgccaccacaaatcattttccccctggtatgaagcacgttgctgggttcgtgcatactggcgcaagcaactttacaaggtaacacagcgtaaggtattaccgaggttaatgaacgaactggaccagcgttattatcttgctgttttgcaggtgtataacgatagccagattcgtaaccacagcagttataacgatgtgcgtgactatattaatgaagtcatgtactcattttcgcgtaaagcgccgaaagaaagttatttggtgatcaaacatcatccgatggatcgtggtcacagactctatcgaccattaattaaacggttgagtaaggaatatggcttaggtgagcgaatcctttatgtgcacgatctcccgatgccggaattattacgccatgcaaaagcggtggtgacgattaacagtacggcggggatctctgcgctgattcataacaaaccactcaaagtgatgggcaatgccctgtacgacatcaagggcttgacgtatcaagggcatttgcaccagttctggcaggctgattttaaaccagatatgaaactgtttaagaagtttcgtgggtatttattggtgaagacgcaggttaatgcggtttattattaa

SEQ ID NO: 34 - DNA, 717 bp, Escherichia coli
atgattgttgcaaatatgtcatcatacccacctcgaaaaaaagagttggtgcattctatacaaagtttacatgctcaagtagataaaattaatctttgcctgaatgagtttgaagaaattcctgaggaattagatggtttttcaaaattaaatccagttattccagataaagattataaggatgtgggcaaatttatatttccttgcgctaaaaatgatatgatcgtacttacagatgatgatattatttaccctcccgattatgtagaaaaaatgctcaatttttataattcctttgcaatattcaattgcattgttgggattcatggctgtatatacatagatgcatttgatggagatcagtctaaaagaaaagtattttcatttactcaagggctattgcgaccgagagttgtaaatcaattaggtacagggactgtttttcttaaggcagatcaattaccatctttaaaatatatggatggttctcaacgattcgtcgatgttagattttctcgctatatgttagagaatgaaattggtatgatatgtgttcccagagaaaaaaactggctaagagaggtctcatcaggttcaatggaaggactttggaacacatttacaaaaaaatggcctttagacatcataaaagaaacacaagcaatcgcaggatattcaaaacttaacctcgaattagtgtataatgtggaagggtaa

SEQ ID NO: 35 - DNA, 1689 bp, Escherichia coli
atgaataaattagtgctagtcggacatcctggctcaaagtatcagatagttgaacattttttgaaagaaattggcatgaactcaccaaattattctacaagtaataaaatttccccagaatatatcaccgcttcattatgtcaattttatcaaacaccagaagttaatgatgtagtagatgagagagaattctcagctgttcaagtctcaaccatgtgggatagcatggttcttgaactaatgatgaacaatctaaataacaaactttgggggtgggcagatccatctataatattttttcttgatttttggaaaaatatagataaaagcataaaattcatcatgatatatgatcaccctaaatataatttaatgcgttcagtaaataatgcccctctctctttaaatataaataatagtgtagataactggattgcatataataaaagattgcttgatttttttttggagaataaagaacgatgtgtgttgattaattttgaggcgtttcaaagcaataagaaaaatattataaagccattgagtaatattataaaaatagataatctaatgtctgcgcattacaaaaattcaatattgtttgatgtggttgagaataatgattatacaaaatcaaatgaaattgccctgcttgaaaaatatacaactttattttctttaagtgcaaatgagactgaaattacatttaatgatacaaaggttagtgagtacttagtatctgaattaataaaagaaagaaccgaggttctgaagctttataatgagttacaagcctatgcaaacctaccttatatagaaacatcgaaagataacgtttcggctgaggctgcattatgggaggtagtcgaagagagaaattctatcttcaatattgtatctcatttggtgcaagagtcaaaaaagaaggatgcagatattgaattgactaaatctatatttaagaaaagacaatttttattattgaacaggattaatgagctaaaaaaagaaaaggaagaggtaattaaactttcaaaaataaatcacaacgatgttgtgagacaagaaaaatatccagatgatattgaaaaaaaaataaatgacatacagaaatatgaagaagagataagcgaaaaagaatcaaaactcactcaggcaatatcagaaaaagaacagattttaaaacaattgcataaatatgaagaagagataagcgaaaaagaatcaaaactcactcaggcaatatcagaaaaagaacagattttaaaacaattgcatatagtgcaagagcagttggaacactattttatagaaaatcaggaaattaaaaagaaacttccacctgtgctatatggagcagctgagcagataaaacaagagttaggttatcgacttggttatattatagtctcgtattctaaatccctcaaggggattattaccatgccatttgcacttatccgtgagtgtgtttttgaaaaaaaacgtaagaagagttatggcgttgatgtgccactctatttatatgctgatgctgataaggctgaaagagttaagaaacatttatcttatcaattagggcaggctattatctccagtgctaattcgatatttggattcattacccttccatttaagttaattgttgttgtttataaatataggagagctaaaatcaagggctgttaa

SEQ ID NO: 36 - DNA, 1563 bp, Escherichia coli
atgaacgcagaatatataaatttagttgaacgtaaaaagaaattagggacaaatattggtgctcttgattttttattatcaattcataaggagaaagttgatcttcaacataaaaactcgcctttaaaaggtaacgataaccttattcacaaaagaataaacgaatacgacaatgtacttgaactatctaagaatgtatcagctcagaattctggcaatgagttttcttatttattgggatatgcagattctcttagaaaagttggtatgttggatacttatattaaaattgtttgttatctaacaattcaatctcgttattttaaaaatggcgaacgagttaagctttttgaacatataagtaacgctctacggtattcaaggagtgattttctcattaatcttatttttgaacgatatatcgaatatataaaccatctaaaattgtcgcccaaacaaaaagatttttatttttgtacgaagttttcaaaatttcatgattatactaaaaatggatataaatatttagcatttgataatcaagccgatgcagggtatggcctgactttattattaaatgcaaacgatgatatgcaagatagttataatctactccctgagcaagaactttttatttgtaatgctgtaatagataatatgaatatttataggagtcaatttaacaaatgtctacgaaaatacgatttatcagaaataactgatatatacccaaataaaattatattgcaaggaattaagttcgataagaaaaaaaatgtttatggaaaagatcttgttagtataataatgtcagtattcaattcagaagatactattgcatactcattacattcattgttgaatcaaacatatgaaaatattgaaattctcgtgtgcgatgattgttcatcggacaaaagccttgaaataattaagagcatagcttattctgattcaagagtgaaagtatatagctcacgaaaaaaccaaggcccttataatataagaaatgagctaataaaaaaagcacacggtaatttcatcacctttcaagatgcagatgatctttctcatccggagagaatacaaagacaagttgaggttcttcgcaataataaggctgtaatctgtatggctaactggatccgtgttgcgtcaaatggaaaaattcaattcttctatgatgataaagccacaagaatgtctgttgtatcgtcaatgataaaaaaagatatttttgcgacagttggtggctatagacaatctttaattggtgcagatacggagttttatgaaacagtaataatgcgttatgggcgagaaagtattgtaagattactgcagccattgatattggggttatggggagactccggacttaccaggaataaaggaacagaagctctacctgatggatatatatcacaatctcgaagagaatatagtgatatcgcggcaagacaacgagtgttagggaaaagtatcgtaagtgataaagatgtacgtggtttattatctcgctatggtttgtttaaagatgtatcaggaataattgaacaatag

SEQ ID NO: 37 - DNA, 1179 bp, Escherichia coli
atgttcggaacactaaaaataactgtttcaggcgctggttacgttgggctttcaaatggaattctaatggctcaaaatcatgaagtggttgcatttgatacccatcaaaaaaaagttgacttacttaatgataaactctctcctatagaggataaggaaattgaaaattatctttcaactaaaatacttaattttcgcgcaactactaacaaatatgaagcctataaaaatgccaattacgttattattgctacaccaacgaattatgacccaggttcaaattactttgatacatcaagcgttgaagctgtcattcgtgacgtaacggaaatcaacccaaacgcaattatggtggttaaatctacggtcccagtaggtttcacaaaaacaattaaagaacatttaggtattaataatattatcttctctccagaatttttacgagaaggaagagccctatacgataatctccatccatctcgcattattatcggtgaatgttctgaacgggcagaacgtttggcagtgttatttcaggaaggagcgattaaacaaaatatacccgttttatttacagattctacggaagcggaagcgattaagttattttcaaatacttatttggctatgcgagttgcattttttaatgaattggatagttacgcagaaagttttggtctgaatacgcgtcagattattgacggtgtttgtttggatccgcgcattggtaattactacaataatccttcttttggttatggtggctactgtttgccaaaagataccaagcaattattagccaactatcagtctgttccgaataaacttatatctgcaattgttgatgctaaccgtacacgtaaggactttatcactaatgttattttgaaacatagaccacaagttgtgggggtttatcgtttgattatgaaaagtggttcagataattttagagattcttctattcttggtattataaagcgtatcaagaaaaaaggcgtgaaagtaattatttatgagccgcttatttctggagatacattctttaactcacctttggaacgggagctggcgatctttaaagggaaagctgatattattatcactaaccgaatgtcagaggagttgaacgatgtggtcgacaaagtctatagtcgcgatttgtttaaatgtgactaa

SEQ ID NO: 38 - DNA, 855 bp, Escherichia coli
atggtactgatgatcgtcagcggacgttcaggttcaggtaaatctgtcgccctgcgtgcgctggaagatatgggtttttactgcgtggataaccttcccgtagtgttgttacccgatctggctcgaactctggccgatcgagagatttctgccgccgtcagcattgatgttcgtaatatgccggagtcaccagaaatattcgaacaggcgatgagtaacctgcctgacgctttctcaccgcaactactgttcctggatgccgaccgtaataccttaattcgtcgttacagtgacacgcgccgactgcatccgctttccagcaaaaacctgtcgctggaaagtgctatcgacaaagaaagcgatttgctggagcctctgcgttcgcgagcggatctgattgtcgacacctcagaaatgtccgttcacgagctggcagaaatgctgcgtacccgtctgctgggtaaacgtgaacgtgaactgaccatggtctttgagtctttcggcttcaaacacggtatccctatcgatgcagattacgtctttgacgtgcgcttcttgccgaacccgcactgggatccgaaactgcgtccaatgacaggtcttgataaacctgtcgccgcgttcctcgaccgccacacagaagtacacaattttatctaccagacgcgaagctatcttgagctatggttacctatgctggaaaccaacaaccgtagctacctgacggtcgccattggttgtaccggcgggaagcaccgttcggtgtatattgcagagcaactggcagactacttccgctcgcgcggtaaaaacgtccagtcacgccatcggacgctggaaaaacgtaaaccatga

(19) United States
(12) Patent Application Publication
(10) Pub. No.: US 2023/0175028 A1
(43) Pub. Date: Jun. 8, 2023

(54) METHOD FOR PRODUCING HEPAROSAN AND BACTERIUM OF GENUS ESCHERICHIA HAVING HEPAROSAN-PRODUCING ABILITY

(71) Applicants: RENSSELAER POLYTECHNIC INSTITUTE, Troy, NY (US); OTSUKA PHARMACEUTICAL FACTORY, INC., Naruto (JP); KIRIN BIOMATERIALS CO., LTD., Tokyo (JP)

(72) Inventors: Ayano KOMINE, Tokyo (JP); Mikiro HAYASHI, Tokyo (JP)

(73) Assignees: RENSSELAER POLYTECHNIC INSTITUTE, Troy, NY (US); OTSUKA PHARMACEUTICAL FACTORY, INC., Naruto (JP); KIRIN BIOMATERIALS CO., LTD., Tokyo (JP)

(21) Appl. No.: 17/995,352

(22) PCT Filed: Apr. 2, 2021
(86) PCT No.: PCT/JP2021/014335

§ 371 Date: Oct. 3, 2022

(30) Foreign Application Priority Data
Apr. 3, 2020 (WO) PCT/JP2020/015384

(51) Int. Cl. C12P 19/26; C12N 15/70; C12N 1/20

(52) U.S. Cl. CPC C12P 19/26; C12N 1/205; C12N 15/70

(57) ABSTRACT

An object of the present invention is to provide a method for efficiently producing heparosan by improving a heparosan-producing ability through genetic modification of a bacterium of the genus Escherichia which has a heparosan-producing ability. The present invention relates to a bacterium of the genus Escherichia which has a genetic modification that increases an expression of a kpsS gene, and which has a heparosan-producing ability; and a method for producing heparosan using the bacterium.

### TECHNICAL FIELD

The present invention relates to a method for producing N-acetylheparosan (hereinafter referred to as heparosan), which is a capsular polysaccharide of a bacterium of the genus Escherichia, by fermentative production using the bacterium.

### BACKGROUND ART

Heparin, which is a sulfated polysaccharide, is an anticoagulant agent, and is used for treating thromboembolism and disseminated intravascular coagulation syndrome, and preventing coagulation during artificial dialysis and in extracorporeal circulation, and the like. Industrially, most heparin in use is extracted and purified from the intestinal mucosa of pigs.

Since a fatal accident occurred in 2008 due to contamination of pig-derived heparin with impurities, research and development of non-animal sourced production-controlled and quality-controlled heparin derived have been required. As a specific example, a method in which fermentatively produced and purified N-acetylheparosan, which is a capsular polysaccharide of Gram-negative microorganisms, is chemically N-deacetylated and N-sulfated, followed by enzymatic epimerization and sulfation to yield heparin having the same structure and anticoagulation activity as those derived from pigs (NPLs 1 and 2).

In the above method, heparosan as a basic structure is a sugar chain composed of a repeating disaccharide structure of glucuronic acid (GlcA) and N-acetyl D-glucosamine (GlcNAc). For production of heparosan, a method of using an Escherichia coli K5 strain originally having a heparosan-producing ability (PTL 1 and NPL 1), a method of using an Escherichia coli Nissle 1917 strain also having a heparosan-producing ability (NPL 2), and a method of using Escherichia coli which does not originally have a heparosan-producing ability (PTL 4 and NPLs 3 and 4) have been reported.

Heparosan is classified as Group 2 capsular polysaccharides, and it is known that gene groups of Region I, Region II, and Region III, which form a cluster on the genome, involves in synthesis and transport of heparosan in an Escherichia coli K5 strain (NPL 4).

It is said that among proteins encoded by those gene groups, KpsS and KpsC allows transfer of a plurality of 3-deoxy-D-manno-octulosonic acid (Kdo) residues to phosphatidylglycerol in the inner membrane, and glycosyltransferases KfiA and KfiC add a precursor sugar nucleotide, thereby synthesis of heparosan proceeds (NPLs 5 and 6).

In addition, KfiD involves in synthesis of a precursor UDP-GlcA; KpsF and KpsU involve in synthesis of CMP-Kdo that is a substrate for Kdo linker synthesis; and KpsM, KpsT, KpsE, and KpsD involve in transport of heparosan synthesized on the inner membrane to the outside of bacterial cells (NPL 6).

As a method of performing heparosan fermentation using BL21 strain and K-12 strain of Escherichia coli which do not have a heparosan-producing ability as hosts, a method of introducing a KfiABCD gene cluster of Region II derived from an Escherichia coli K5 strain into the host is known. In addition, genes that improve heparosan production, such as rfaH, nusG, and rpoE, have been reported (PTL 4, and NPLs 3 and 4).

### Citation List

### Patent Literature

PTL 1: Japanese Patent No. 5830464
PTL 2: U.S. Pat. No. 8771995
PTL 3: WO2018/048973
PTL 4: U.S. Pat. No. 9975928

### Non-Patent Literature

NPL 1: Biotechnology and Bioengineering 107 (2010) 964-973
NPL 2: Applied Microbiology and Biotechnology 103 (2019) 6771-6782
NPL 3: Metabolic Engineering 14 (2012) 521-527
NPL 4: Carbohydrate Research 360 (2012) 19-24
NPL 5: Proceedings of the National Academy of Sciences of USA 110 (2013) 20753-20758
NPL 6: Carbohydrate Research 378 (2013) 35-44

### SUMMARY OF INVENTION

### Technical Problem

As described above, research and development for production of production-controlled and quality-controlled heparin derived from non-animals have been conducted, but the conventional production methods were insufficient in efficiency. Meanwhile, how each of genes encoded by the Regions I, II, and III affect heparosan production by bacteria of the genus Escherichia which have a heparosan-producing ability is not known until now.

Accordingly, an object of the present invention is to provide a method for efficiently producing heparosan by improving efficiency of producing heparosan through genetic modification of a bacterium of the genus Escherichia which has an ability to produce heparosan.

### Solution to Problem

The inventors of the present invention have found that heparosan production efficiency is improved by using a bacterium of the genus Escherichia which has a specific genetic modification and has a heparosan-producing ability, and therefore have completed the present invention.

That is, the present invention is as follows.
1. A method for producing heparosan, comprising: culturing a bacterium of the genus Escherichia which has the following genetic modification (1) and has a heparosan-producing ability in a medium to produce heparosan in the medium:
(1) a genetic modification increasing an expression of a kpsS gene.
2. The method for producing heparosan according to 1, wherein the bacterium of the genus Escherichia further has at least one of the following genetic modifications (2) and (3):
(2) a genetic modification increasing an expression of at least one gene selected from a kfiA gene, a kfiB gene, a kfiC gene, and a kfiD gene, and
(3) a genetic modification causing loss of a function of a yhbJ gene.
3. The method for producing heparosan according to 1 or 2, wherein the genetic modification (1) is at least one of modifying an expression control region of the kpsS gene and increasing the copy number of the kpsS gene.
4. The method for producing heparosan according to 2 or 3, wherein the genetic modification (2) is at least one of modifying an expression control region of at least one gene selected from the kfiA gene, the kfiB gene, the kfiC gene, and the kfiD gene, and increasing the copy number of at least one gene selected from the kfiA gene, the kfiB gene, the kfiC gene, and the kfiD gene.
5. The method for producing heparosan according to any one of 2 to 4, wherein the genetic modification (3) is deletion of the yhbJ gene.
6. The method for producing heparosan according to any one of 1 to 5, wherein the bacterium of the genus Escherichia is Escherichia coli.
7. The method for producing heparosan according to any one of 1 to 6, wherein the kpsS gene is a DNA comprising the nucleotide sequence shown in SEQ ID NO: 33, or a DNA comprising a nucleotide sequence that has 90% or more identity to the nucleotide sequence shown in SEQ ID NO: 33 and having a property of increasing a heparosan-producing ability of the bacterium of the genus Escherichia having the heparosan-producing ability when an expression level is increased in the bacterium.
8. The method for producing heparosan according to any one of 2 to 7, wherein the kfiA gene is a DNA comprising the nucleotide sequence shown in SEQ ID NO: 34, or a DNA comprising a nucleotide sequence that has 90% or more identity to the nucleotide sequence shown in SEQ ID NO: 34 and having a property of increasing a heparosan-producing ability of the bacterium of the genus Escherichia having the heparosan-producing ability when an expression level is increased in the bacterium, the kfiB gene is a DNA comprising the nucleotide sequence shown in SEQ ID NO: 35, or a DNA comprising a nucleotide sequence that has 90% or more identity to the nucleotide sequence shown in SEQ ID NO: 35 and having a property of increasing a heparosan-producing ability of the bacterium of the genus Escherichia having the heparosan-producing ability when an expression level is increased in the bacterium, the kfiC gene is a DNA comprising the nucleotide sequence shown in SEQ ID NO: 36, or a DNA comprising a nucleotide sequence that has 90% or more identity to the nucleotide sequence shown in SEQ ID NO: 36 and having a property of increasing a heparosan-producing ability of the bacterium of the genus Escherichia having the heparosan-producing ability when an expression level is increased in the bacterium, and
the kfiD gene is a DNA comprising the nucleotide sequence shown in SEQ ID NO: 37, or a DNA comprising a nucleotide sequence that has 90% or more identity to the nucleotide sequence shown in SEQ ID NO: 37 and having a property of increasing a heparosan-producing ability of the bacterium of the genus Escherichia having the heparosan-producing ability when an expression level is increased in the bacterium.
9. The method for producing heparosan according to any one of 2 to 8, wherein the yhbJ gene is a DNA comprising the nucleotide sequence shown in SEQ ID NO: 38, or a DNA comprising a nucleotide sequence that has 90% or more identity to the nucleotide sequence shown in SEQ ID NO: 38 and having a property of increasing a heparosan-producing ability of the bacterium of the genus Escherichia having the heparosan-producing ability when an expression level is decreased in the bacterium.
10. The method for producing heparosan according to any one of 1 to 9, wherein the bacterium of the genus Escherichia does not have the following genetic modification (4):
(4) a genetic modification increasing an expression of a kpsC gene.
11. A bacterium of the genus Escherichia which has a heparosan-producing ability and has the following genetic modification (1):
(1) a genetic modification increasing an expression of a kpsS gene.
12. The bacterium of the genus Escherichia according to 11, which further has at least one of the following genetic modifications (2) and (3):
(2) a genetic modification increasing an expression of at least one gene selected from a kfiA gene, a kfiB gene, a kfiC gene, and a kfiD gene, and
(3) a genetic modification causing loss of a function of a yhbJ gene.
13. The bacterium of the genus Escherichia according to 11 or 12, which does not have the following genetic modification (4):
(4) a genetic modification increasing an expression of a kpsC gene.

### Advantageous Effects of Invention

According to a method for producing heparosan of the present invention, heparosan derived from non-animals can be produced with excellent production efficiency by using a bacterium of the genus Escherichia which has a specific genetic modification and has a heparosan-producing ability.

### BRIEF DESCRIPTION OF DRAWINGS

FIG. 1 shows a schematic diagram of a heparosan synthesis gene cluster on the chromosome of an Escherichia coli K5 strain.

FIG. 2 shows a schematic diagram of an enzyme involved in heparosan production.

FIG. 3 shows a schematic diagram of a heparosan biosynthetic pathway.

### DESCRIPTION OF EMBODIMENTS

Hereinafter, terms used in the present specification have meanings generally used in the field unless otherwise specified.

### Bacterium of the Present Invention

In a method for producing heparosan of the present invention, a bacterium of the genus Escherichia which has the following genetic modification (1) and has a heparosan-producing ability (hereinafter abbreviated as the bacterium of the present invention) is used:

a genetic modification increasing an expression of a kpsS gene.

The bacterium of the present invention preferably further has at least one of the following genetic modifications (2) and (3):
(2) a genetic modification increasing an expression of at least one gene selected from a kfiA gene, a kfiB gene, a kfiC gene, and a kfiD gene, and
(3) a genetic modification causing loss of a function of a yhbJ gene.
Accordingly, examples of genetic modifications in the bacterium of the genus Escherichia include (1) and (2) described above, (1) and (3) described above, and (1) to (3) described above.

The bacterium of the present invention preferably does not have the following genetic modification (4):

a genetic modification increasing an expression of a kpsC gene.

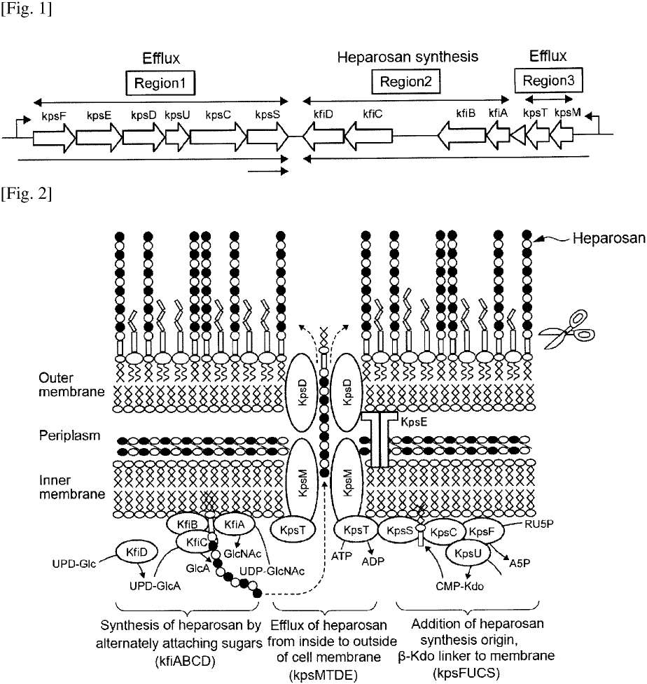

As an example of a bacterium of the genus Escherichia, FIG. 1 shows a schematic diagram of a heparosan synthesis gene cluster on the chromosome of an Escherichia coli K5 strain [J. Nzakizwanayo et al., PLOS ONE, (2015)]. In addition, FIG. 2 shows a schematic diagram of an enzyme involved in heparosan production.

As shown in FIG. 1, the kpsS and kpsC are genes encoded by Region I among the gene group of Region I, Region II, and Region III. As shown in FIG. 2, the kpsS and kpsC are involved in initiation of heparosan synthesis. In the heparosan production, kpsS, together with kpsC, plays a role in adding multiple Kdo linkers to phosphatidylglycerol in the inner membrane.

As the kpsS gene, a kpsS gene derived from the genus Escherichia is preferable. Specific examples thereof include a kpsS gene of an Escherichia coli K5 strain. A nucleotide sequence of the kpsS gene of the Escherichia coli K5 strain and an amino acid sequence of a protein encoded by the gene can be obtained from public databases. The kpsS gene of the Escherichia coli K5 strain is registered as GenBank accession CAA52659.1.

Examples of kpsS genes include a DNA comprising the nucleotide sequence shown in SEQ ID NO: 33, or a DNA comprising a nucleotide sequence that has 90% or more identity to the nucleotide sequence shown in SEQ ID NO: 33 and having a property of increasing a heparosan-producing ability of the bacterium of the genus Escherichia having the heparosan-producing ability when an expression level is increased in the bacterium.

As shown in FIG. 1, the kfiA, kfiB, kfiC, and kfiD are genes encoded by Region II among the gene group of Region I, Region II, and Region III. As shown in FIG. 2, the kfiA, kfiB, kfiC and kfiD are involved in synthesis of heparosan, and play a role of adding saccharide and thereby synthesizing heparosan.

As the kfiA, kfiB, kfiC or kfiD gene, a kfiA, kfiB, kfiC, or kfiD gene derived from the genus Escherichia is preferable. Specific examples thereof include a kfiA, kfiB, kfiC, or kfiD gene of an Escherichia coli K5 strain. A nucleotide sequence of the kfiA, kfiB, kfiC, or kfiD gene of the Escherichia coli K5 strain and an amino acid sequence of a protein encoded by the gene can be obtained from public databases. The kfiA is registered as GenBank accession CAA54711.1; the kfiB is registered as GenBank accession CAE55824.1; the kfiC is registered as GenBank accession CAA54709.1; and the kfiD is registered as GenBank accession CAA54708.1.

Examples of kfiA genes include a DNA comprising the nucleotide sequence shown in SEQ ID NO: 34, or a DNA comprising a nucleotide sequence that has 90% or more identity to the nucleotide sequence shown in SEQ ID NO: 34 and having a property of increasing a heparosan-producing ability of the bacterium of the genus Escherichia having the heparosan-producing ability when an expression level is increased in the bacterium.

Examples of kfiB genes include a DNA comprising the nucleotide sequence shown in SEQ ID NO: 35, or a DNA comprising a nucleotide sequence that has 90% or more identity to the nucleotide sequence shown in SEQ ID NO: 35 and having a property of increasing a heparosan-producing ability of the bacterium of the genus Escherichia having the heparosan-producing ability when an expression level is increased in the bacterium.

Examples of kfiC genes include a DNA comprising the nucleotide sequence shown in SEQ ID NO: 36, or a DNA comprising a nucleotide sequence that has 90% or more identity to the nucleotide sequence shown in SEQ ID NO: 36 and having a property of increasing a heparosan-producing ability of the bacterium of the genus Escherichia having the heparosan-producing ability when an expression level is increased in the bacterium.

Examples of kfiD genes include a DNA comprising the nucleotide sequence shown in SEQ ID NO: 37, or a DNA comprising a nucleotide sequence that has 90% or more identity to the nucleotide sequence shown in SEQ ID NO: 37 and having a property of increasing a heparosan-producing ability of the bacterium of the genus Escherichia having the heparosan-producing ability when an expression level is increased in the bacterium.

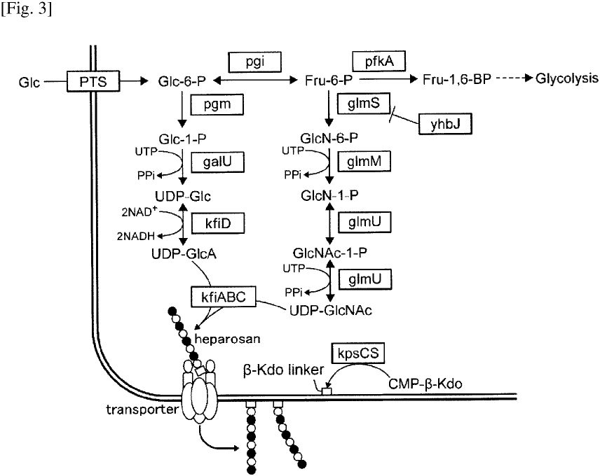

FIG. 3 shows a schematic diagram of a heparosan biosynthetic pathway. As shown in FIG. 3, GlmS is the first enzyme in an UDP-N-acetylglucosamine supply pathway that is a precursor of heparosan and is an enzyme that catalyzes reaction from fructose-6-phosphate to glucosamine-6-phosphate. YhbJ is an enzyme that negatively controls GlmS.

As the yhbJ gene, a yhbJ gene derived from the genus Escherichia is preferable. Specific examples thereof include a yhbJ gene of an Escherichia coli K-12 strain. A nucleotide sequence of the yhbJ gene of the Escherichia coli K-12 strain and an amino acid sequence of a protein encoded by the gene can be obtained from public databases. The yhbJ gene of the Escherichia coli K-12 strain is registered as GenBank accession BAE77249.1.

Examples of yhbJ genes include a DNA comprising the nucleotide sequence shown in SEQ ID NO: 38, or a DNA comprising a nucleotide sequence that has 90% or more identity to the nucleotide sequence shown in SEQ ID NO: 38 and having a property of increasing a heparosan-producing ability of the bacterium of the genus Escherichia having the heparosan-producing ability when an expression level is decreased in the bacterium.

Each of the genes in (1) to (3) above can be easily obtained from public databases by, for example, a BLAST search or a FASTA search using a nucleotide sequence of each gene described above. In addition, a homologue of each gene can be obtained by, for example, PCR using a chromosome of a microorganism such as a bacterium as a template and using an oligonucleotide produced based on these known gene sequences as a primer.

Each of the genes in (1) to (3) above may be a variant of the genes as long as original functions (for example, an activity or a property) of a protein encoded by the genes are maintained. Whether or not a protein encoded by a variant of the genes maintains its original function can be checked; specifically, for example, when the original function is to improve a heparosan-producing ability, by introducing the variant of the gene into a microorganism belonging to prokaryotes having the heparosan-producing ability.

Variants of each of the genes in (1) to (3) above can be obtained according to a site-directed mutagenesis method by modifying an encoding region of a gene such that amino acid residues at specific positions of an encoded protein are substituted, deleted, inserted, or added. In addition, variants of each of the genes in (1) to (3) above can also be obtained by, for example, mutation treatment.

As long as their original functions are maintained, each of the genes (1) to (3) above may be genes encoding a protein having an amino acid sequence in which one or several amino acids at one or several positions are substituted, deleted, inserted, or added. For example, in an encoded protein, its N-terminus and/or C-terminus may be extended or shortened. The phrase “one or several” differs depending on the position and type of an amino acid residue in the three-dimensional structure of a protein. Specific examples thereof include 1 to 50, 1 to 40, 1 to 30, and it is preferably 1 to 20, more preferably 1 to 10, even more preferably 1 to 5, and particularly preferably 1 to 3.

The substitution, deletion, insertion, or addition of one or several amino acids as described above is a conservative mutation that maintains a function of a protein normally. Representatives of conservative mutations are a conservative substitution. The conservative substitution is a mutation in which substitution occurs between Phe, Trp, and Tyr in a case where a substitution site is an aromatic amino acid, substitution occurs between Leu, Ile, and Val in a case where a substitution site is a hydrophobic amino acid, substitution occurs between Gln and Asn in a case where a substitution site is a polar amino acid, substitution occurs between Lys, Arg, and His in a case where a substitution site is a basic amino acid, substitution occurs between Asp and Glu in a case where a substitution site is an acidic amino acid, and substitution occurs between Ser and Thr in a case where a substitution site is an amino acid having hydroxyl groups. Specific examples of substitutions considered as a conservative substitution include substitution of Ala with Ser or Thr; substitution of Arg with Gln, His, or Lys; substitution of Asn with Glu, Gln, Lys, His, or Asp; substitution of Asp with Asn, Glu, or Gln; substitution of Cys with Ser or Ala; Substitution of Gln with Asn, Glu, Lys, His, Asp, or Arg; substitution of Glu with Gly, Asn, Gln, Lys, or Asp; substitution of Gly with Pro; substitution of His with Asn, Lys, Gln, Arg, or Tyr; substitution of Ile with Leu, Met, Val, or Phe; substitution of Leu with Ile, Met, Val, or Phe; substitution of Lys with Asn, Glu, Gln, His, or Arg; substitution of Met with Ile, Leu, Val, or Phe; substitution of Phe with Trp, Tyr, Met, Ile, or Leu; substitution of Ser with Thr or Ala; substitution of Thr with Ser or Ala; substitution of Trp with Phe or Tyr; substitution of Tyr with His, Phe, or Trp; and substitution of Val with Met, Ile, or Leu. In addition, the substitutions, deletions, insertions, additions of amino acids as described above, inversions thereof, and the like include substitutions, deletions, insertions, and additions, inversions, and the like which are caused by mutations (mutants or variants) that occur naturally such as mutations based on individual differences or species differences in an organism from which a gene is derived.

In addition, as long as their original functions are maintained, each of the genes in (1) to (3) above may be genes encoding a protein that has 80% or more, preferably 90% or more, more preferably 95% or more, even more preferably 97% or more, and particularly preferably 99% or more identity to the entire amino acid sequence.

In addition, as long as their original functions are maintained, each of the genes in (1) to (3) above may be a DNA that hybridizes, under stringent conditions, with a probe that can be prepared from known gene sequences, such as a sequence complementary to the whole or a part of the nucleotide sequence. The term “stringent conditions” refers to conditions under which so-called specific hybrids are formed and non-specific hybrids are not formed. Examples thereof include conditions under which DNAs having identity to each other at a higher level, for example, DNAs that have 80% or more, preferably 90% or more, more preferably 95% or more, even more preferably 97% or more, and particularly preferably 99% or more identity to each other hybridize with each other, and DNAs having identity to each other at a lower level do not hybridize with each other; or conditions, which are conditions for washing in a normal Southern hybridization, in which washing is performed once, preferably two to three times at a salt concentration and a temperature corresponding to 60° C., 1 x SSC, and 0.1% SDS, preferably 60° C., 0.1 x SSC, and 0.1% SDS, and more preferably 68° C., 0.1 x SSC, and 0.1% SDS.

The probe used for the above hybridization may be a part of the complementary sequence of each gene. Such a probe can be produced by PCR using an oligonucleotide produced based on a known gene sequence as a primer and using a DNA fragment containing each of the genes in (1) to (3) above as a template. For example, a DNA fragment having a length of about 300 bp can be used as a probe. In a case where a DNA fragment having a length of about 300 bp is used as a probe, examples of conditions for washing in hybridization include conditions of 50° C., 2 x SSC, and 0.1% SDS.

In addition, because codon degeneracy differs depending on hosts, each of the genes in (1) to (3) above may be genes obtained by substituting any codon by an equivalent codon as long as their original functions are maintained. For example, genes in Tables 1 to 3 may be modified so that they have optimal codons depending on frequency of codon usage of a host used.

Examples of mutation treatments include a method of in vitro treating a DNA molecule having a nucleotide sequence of each of the genes in (1) to (3) above with hydroxylamine or the like; a method of treating microorganisms carrying each of the genes in (1) to (3) above with X-rays, ultraviolet rays, or a mutagen such as N-methyl-N′-nitro-N-nitrosoguanidine (NTG), ethyl methanesulfonate (EMS), and methyl methanesulfonate (MMS), and the like.

### Genetic Modification to Increase Gene Expression Level

The phrase “increase in gene expression” means that an expression of a gene is elevated as compared to that of an unmodified strain. Examples of one aspect of increasing expression of a gene include an aspect in which expression of a gene is preferably increased 1.5-fold or more, is more preferably increased 2-fold or more, and is even more preferably increased 3-fold or more, as compared to that of an unmodified strain.

In addition, the phrase “gene expression is increased” means not only an increase in expression of a target gene in a strain in which the target gene is originally expressed, but also means that a target gene is expressed in a strain in which the target gene is originally not expressed. That is, the phrase “gene expression is increased” includes, for example, a case in which a target gene is introduced into a strain not having the target gene, and the target gene is expressed therein. Furthermore, the phrase “gene expression is increased” is also referred to as the phrases “gene expression is enhanced” and “gene expression is elevated.”

An increase in gene expression can be achieved by, for example, increasing the copy number of the gene. Increasing the copy number of the gene can be achieved by introducing the gene into the chromosome of a host. Introduction of the gene into the chromosome can be performed using, for example, homologous recombination (Miller I, J.H. Experiments in Molecular Genetics, 1972, Cold Spring Harbor Laboratory). Only one copy of the gene may be introduced, or two or more copies thereof may be introduced.

For example, multiple copies of a gene can be introduced into the chromosome by performing homologous recombination while targeting a sequence having multiple copies on the chromosome. Examples of the sequence having multiple copies on the chromosome include a repetitive DNA sequence (a repetitive DNA), and inverted repeats present at both ends of a transposon.

Alternatively, homologous recombination may be performed while targeting an appropriate sequence on the chromosome, such as a gene unnecessary for production of a target substance. The homologous recombination can be performed by, for example, a method using a linear DNA, a method using a plasmid containing a temperature-sensitive replication origin, a method using a plasmid capable of conjugative transfer, a method using a suicide vector not having a replication origin that functions in a host, or a transduction method using phage. In addition, a gene can also be randomly introduced into the chromosome using a transposon or Mini-Mu (JP-A-H2-109985).

Whether a target gene has been introduced into the chromosome can be checked by Southern hybridization using a probe having a sequence complementary to all or a part of the gene, PCR using a primer created based on a sequence of the gene, or the like.

In addition, the copy number of gene can also be increased by introducing a vector containing the gene into a host. For example, it is possible to increase the copy number of the gene by constructing an expression vector for the gene by ligating a DNA fragment containing the target gene to a vector that functions in a host, and transforming the host with the expression vector. The DNA fragment containing the target gene can be obtained by, for example, PCR using a genomic DNA of a microorganism having the target gene as a template. A transformation method is not particularly limited, and a conventionally known method can be used.

As the vector, a vector that can autonomously replicate in a host cell can be used. The vector is preferably a multicopy vector. In addition, the vector preferably has a marker such as an antibiotic resistance gene or another gene described in the literature [Karl Friehs, Plasmid Copy Number and Plasmid Stability, Adv Biochem Engin/ Biotechnol 86: 47-82 (2004)] in order to select a transformant. Furthermore, the vector may have a promoter or a terminator in order to express an inserted gene. Examples of the vector include a vector derived from a bacterial plasmid, a vector derived from a yeast plasmid, a vector derived from a bacteriophage, a cosmid, a phagemid, and the like.

Specific examples of vectors capable of autonomous replication in bacteria of Enter-obacteriaceae such as Escherichia coli include pUC19, pUC18, pHSG299, pHSG399, pHSG398, pBR322, and pSTV29 (all from Takara Bio Inc.), pACYC184, pMW219, pMW118 and pMW119 (all from Nippon Gene), pTrc99A (Pharmacia), a pPROK vector (Clontech), pKK233-2 (Clontech), a pET vector (Novagen), a pQE vector (Qiagen), and a broad host range vector RSF1010.

In a case of introducing a gene, it is sufficient for the gene be retained in a bacterium of the genus Escherichia having the genetic modification in the present invention. Specifically, it is sufficient for the gene be introduced such that it is expressed upon control of a promoter sequence that functions in the bacterium of the present invention. A promoter may be a promoter derived from a host or a heterologous promoter. The promoter may be an intrinsic promoter of a gene to be introduced or a promoter of other genes. As the promoter, for example, a stronger promoter which will be described later may be used.

A terminator for terminating transcription can be disposed downstream of a gene. The terminator is not particularly limited as long as it functions in the bacterium of the present invention. The terminator may be a terminator derived from a host or a heterologous terminator. The terminator may be a terminator specific to a gene to be introduced, or may be a terminator of other genes. Specific examples of terminators include a T7 terminator, a T4 terminator, an fd phage terminator, a tet terminator, and a trpA terminator.

Vectors, promoters, and terminators that can be used in various microorganisms are described in detail in, for example, “Basic Lecture 8 on Microbiology, Gene Engineering, KYORITSU SHUPPAN, 1987,” and these can be used.

In addition, in a case where two or more genes are introduced, it is sufficient for each gene be retained in the bacterium of the present invention in an expressible manner. For example, each gene may all be retained on a single expression vector, or all may be retained on a chromosome. Furthermore, each gene may be separately retained on a plurality of expression vectors, or may be separately retained on a single or a plurality of expression vectors and on a chromosome. Furthermore, two or more genes may form an operon and be introduced. Examples of the “cases where two or more genes are introduced” include a case of introducing genes respectively encoding two or more enzymes, a case of introducing genes respectively encoding two or more subunits that form a single enzyme, and combinations thereof.

A gene to be introduced is not particularly limited as long as it encodes a protein that functions in a host. The gene to be introduced may be a gene derived from a host or a heterologous gene. The gene to be introduced can be obtained by, for example, PCR using primers designed based on a nucleotide sequence of the gene and using genomic DNA of an organism having the gene or a plasmid or the like carrying the gene as a template. In addition, the gene to be introduced may be totally synthesized, for example, based on a nucleotide sequence of the gene [Gene, 60 (1), 115-127 (1987)].

Furthermore, an increase in a gene expression can be achieved by improving transcription efficiency of the gene. Improving transcription efficiency of a gene can be achieved by, for example, substituting a promoter of the gene on the chromosome by a stronger promoter. The “stronger promoter” refers to a promoter that enhances gene transcription over a naturally occurring wild-type promoter.

Examples of the “stronger promoters” include known high expression promoters such as a uspA promoter, a T7 promoter, a trp promoter, a lac promoter, a thr promoter, a tac promoter, a trc promoter, a tet promoter, an araBAD promoter, an rpoH promoter, a PR promoter, and a PL promoter.

In addition, as the stronger promoter, a conventional promoter of a highly active type may be obtained using various reporter genes. For example, an activity of the promoter can be increased by bringing -35 and -10 regions in a promoter region closer to a consensus sequence (WO2000/18935).

Examples of highly-active-type promoters include various tac-like promoters (Katashkina JI et al. Russian Federation Patent application 2006134574) and a pnlp8 promoter (WO2010/027045). Methods for evaluating promoter strength, and examples of strong promoters are described in known literature [Prokaryotic promoters in biotechnology. Biotechnol. Annu. Rev., 1, 105-128 (1995) and the like].

In addition, an increase in a gene expression level can be achieved by improving translation efficiency of the gene. Improvement in translation efficiency of a gene can be achieved by, for example, substituting a Shine-Dalgarno (SD) sequence (also called a ribosome binding site (RBS)) of the gene on the chromosome by a stronger SD sequence.

The “stronger SD sequence” refers to an SD sequence in which mRNA translation is improved over a naturally occurring wild-type SD sequence. Examples of the stronger SD sequence include the RBS of gene 10 from phage T7 (Olins P.O. et al, Gene, 1988, 73, 227-235). In addition, substitution, insertion, or deletion of several nucleotides in a spacer region between an RBS and a start codon, particularly in a sequence immediately upstream of the start codon (5′-UTR), is known to significantly affect stability and translation efficiency of mRNA, and therefore, translation efficiency of a gene can be improved by modifying these.

In the present invention, sites that affect expression of a gene, such as a promoter, an SD sequence, and a spacer region between an RBS and a start codon, are also collectively referred to as “expression control regions.” The expression control regions can be determined using a gene search software such as a promoter search vector or GENETYX. Modification of these expression control regions can be performed by, for example, a method using a temperature-sensitive vector or a Red driven integration method (WO2005/010175).

Improvement in gene translation efficiency can also be achieved by, for example, codon modification. Specifically, for example, in a case where heterologous expression of a gene is performed, and the like, translation efficiency of the gene can be improved by replacing rare codons present in the gene with synonymous codons used more frequently.

Codon substitution can be performed by, for example, a site-directed mutagenesis method in which a target mutation is introduced into a target site in a DNA. Examples of the site-directed mutagenesis methods include a method using PCR [Higuchi, R., 61, in PCR technology, Erlich, H.A. Eds., Stockton press (1989); Carter, P., Meth. In Enzymol., 154, 382 (1987)], and a method using phage [Kramer, W. and Frits, H.J., Meth. In Enzymol., 154, 350 (1987); Kunkel, T.A. et al., Meth. In Enzymol., 154, 367 (1987)]. Alternatively, a gene fragment in which codons have been substituted may be totally synthesized. A frequency of codon usage in various organisms is described in “Codon usage database” [http://www.kazusa.or.jp/codon; Nakamura, Y. et al, Nucl. Acids Res., 28, 292 (2000)].

In addition, expression of a gene can also be increased by amplifying a regulator that increases expression of a gene, or by deleting or weakening a regulator that decreases expression of a gene. Such techniques for increasing expression of a gene as described above may be used alone or may be used in any combination.

An increase in expression of gene can be checked, for example, by checking an increase in transcription amount of the gene or by checking an increase in an amount of a protein expressed from the gene. In addition, an increase in expression of a gene can be checked, for example, by checking an increase activity of a protein expressed from the gene.

Checking an increase in transcription amount of a gene can be performed by comparing an amount of mRNA transcribed from the gene with an unmodified strain such as a wild strain or a parent strain. Examples of methods for evaluating an amount of mRNA include Northern hybridization, RT-PCR, and the like [Sambrook, J., et al., Molecular Cloning A Laboratory Manual/Third Edition, Cold spring Harbor Laboratory Press, Cold spring Harbor (USA), 2001]. An increase in an amount of mRNA refers to, for example, the case in which an amount of mRNA has preferably increased 1.5-fold or more, has more preferably increased 2-fold or more, and has even more preferably increased 3-fold or more, as compared to that of an unmodified strain.

An increase in amount of protein can be checked by, for example, Western blot using an antibody. An increase in amount of protein refers to, for example, the case in which an amount of protein has preferably increased 1.5-fold or more, has more preferably increased 2-fold or more, and has even more preferably increased 3-fold or more, as compared to that of an unmodified strain.

An increase in an activity of a protein can be checked by, for example, measuring the activity of the protein. The increase in the activity of the protein refers to, for example, the case in which an activity of a protein has preferably increased 1.5-fold or more, has more preferably increased 2-fold or more, and has even more preferably increased 3-fold or more, as compared to that of an unmodified strain.

The above-described techniques for increasing expression of a gene can be used for enhancing expression of each of the genes (1) and (2) above.

Genetic modification that increases expression of the kpsS gene is preferably at least one of modification of expression control regions of the kpsS gene and genetic modification that increases the copy number. A kpsFEDUCS gene is present as a heparosan-producing gene group, but as will be described later in the Examples, the inventors of the present invention have found that an effect of particularly improving production of heparosan is obtained by increasing expression of only the kpsS gene among them. Accordingly, as the genetic modification that increases expression of the kpsS gene, a genetic modification for increasing the copy number of the kpsS gene is particularly preferable.

While the bacterium of the present invention has the genetic modification that increases expression of the kpsS gene, the bacterium of the present invention preferably does not have a genetic modification that increases an expression of kpsC gene, more preferably does not have a genetic modification that increases an expression of kpsC gene and at least one gene selected from kpsF, kpsE, kpsD and kpsU genes, and most preferably does not have a genetic modification that increases an expression of all the kpsC, kpsF, kpsE, kpsD and kpsU genes.

A genetic modification that increases expression of at least one gene selected from the kfiA, kfiB, kfiC, and kfiD genes is preferably at least one of a modification of expression control regions of at least one gene selected from the kfiA, kfiB, kfiC, and kfiD genes, and increasing the copy number of the genes. The kfiA, kfiB, kfiC, and kfiD genes constitute an operon as shown in FIG. 1. A genetic modification that enhances the entire operon constituted by the kfiA, kfiB, kfiC, and kfiD genes is preferable, and a modification of expression control regions of the kfiA, kfiB, kfiC, and kfiD genes is more preferable.

### Genetic Modification That Causes Loss of Gene Function

Examples of genetic modifications that causes loss of function of the yhbJ gene of (3) above include a genetic modification in which a function of a protein encoded by a part corresponding to yhbJ which causes loss of function of the yhbJ gene is reduced or completely stopped by modifying a DNA encoding the part corresponding to yhbJ in a genomic DNA of a bacterium of the genus Escherichia as a host.

In the method of the present invention, a form of modification to be added to the DNA encoding the part corresponding to yhbJ is not particularly limited as long as a function of a protein encoded by the part corresponding to yhbJ is reduced or completely stopped, and a known method can be appropriately used.

Examples of forms that reduces or completely stops a function of a protein encoded by the part corresponding to yhbJ include any one of the following modifications (a) to (c):
(a) All or a part of the DNA encoding the part corresponding to yhbJ is removed,
(b) One or several substitutions, deletions, or additions are made to the DNA encoding the part corresponding to yhbJ, and
(c) The DNA encoding the part corresponding to yhbJ is substituted by a DNA sequence having less than 80% of identity to a DNA sequence before modification.

Examples of loss of a function of the yhbJ gene include the case in which an activity of yhbJ gene is preferably 20% or less, more preferably 10% or less, and even more preferably 5% or less, as compared with that of an unmodified strain. An activity of yhbJ can be checked by examining an expression level of glmS by a Northern blotting method, a Western blotting method, or the like [Kalamorz F. et al, (2007) “Feedback control of glucosamine-6-phosphate synthase GlmS expression depends on the small RNA GlmZ and involves the novel protein YhbJ in Escherichia coli.” Mol Microbiol. 65 (6):1518-33].

### Bacterium of the Genus Escherichia

The bacterium belonging to the genus Escherichia is not particularly limited, and examples thereof include a bacterium classified into the genus Escherichia by a classification known to experts in microbiology. Examples of bacteria belonging to the genus Escherichia include bacteria described in the literature [Backmann, B.J. 1996. Derivations and Genotypes of some mutant derivatives of Escherichia coli K-12, p. 2460-2488. Table 1. In F.D. Neidhardt (ed.), Escherichia coli and Salmonella Cellular and Molecular Biology/Second Edition, American Society for Microbiology Press, Washington, DC].

Examples of bacteria belonging to the genus Escherichia include Escherichia coli. Examples of Escherichia coli include Escherichia coli K-12 strains such as a W3110 strain (ATCC 27325) and a MG1655 strain (ATCC 47076); an Escherichia coli K5 strain (ATCC 23506); Escherichia coli B strains such as a BL21 (DE3) strain; an Escherichia coli Nissle 1917 strain (DSM 6601); and derivative strains thereof.

These strains can be ordered from, for example, the American Type Culture Collection (address: 12301 Parklawn Drive, Rockville, Maryland 20852, P.O. Box 1549, Manassas, VA 20108, United States of America). That is, a registration number is given to each strain, and the strains can be ordered using this registration number (refer to https://www.atcc.org/). A registration number corresponding to each strain is described in the catalog of the American Type Culture Collection. In addition, the BL21 (DE3) strain is available from, for example, Life Technologies (product number C6000-03).

The bacterium of the present invention may originally have a heparosan-producing ability, or may have been modified to have a heparosan-producing ability. A bacterium having a heparosan-producing ability can be obtained by, for example, imparting a heparosan-producing ability to the above-mentioned bacterium.

The heparosan-producing ability can be imparted by introducing a gene encoding a protein involved in heparosan production by referring to Metabolic Engineering 14 (2012) 521-527, Carbohydrate Research 360 (2012) 19-24, U.S. Pat. No. 9,975,928, and the like Examples of proteins involved in heparosan production include a glycosyl-transferase and a heparosan-efflux carrier protein. In the present invention, one kind of gene may be introduced, or two or more kinds of genes may be introduced. Introduction of a gene can be performed in the same manner as in the above-described method for increasing the copy number of gene.

### Method for Producing Heparosan

The method for producing heparosan of the present invention comprising culturing the bacterium of the present invention in a medium to produce and accumulate heparosan in the medium. The method for producing heparosan of the present invention may further comprises, if necessary, collecting heparosan from the medium.

A medium used is not particularly limited as long as the bacterium of the present invention can grow and heparosan is produced and accumulated. As the medium, for example, a normal medium used for culturing bacteria can be used. Examples of media include a LB medium (a Luria-Bertani medium), but examples are not limited thereto. As the medium, it is possible to use, for example, a medium containing a component selected from a carbon source, a nitrogen source, a phosphate source, a sulfur source, and other various organic components and inorganic components as necessary. Those skilled in the art may appropriately set the type and a concentration of medium components.

The carbon source is not particularly limited as long as it can be utilized by the bacterium of the present invention so that heparosan can be produced. Examples of carbon sources include sugars such as glucose, fructose, sucrose, lactose, galactose, xylose, arabinose, molasses, starch hydrolyzate, and biomass hydrolyzate, organic acids such as acetic acid, fumaric acid, citric acid, succinic acid, and malic acid, alcohols such as glycerol, crude glycerol, and ethanol, and fatty acids. As the carbon source, one kind of carbon source may be used, or two or more kinds of carbon sources may be combined.

Examples of nitrogen sources include ammonium salts such as ammonium sulfate, ammonium chloride, and ammonium phosphate, organic nitrogen sources such as peptone, yeast extract, meat extract, and soy protein decomposed products, ammonia, and urea. As the nitrogen source, one kind of nitrogen source may be used, or two or more kinds of nitrogen sources may be combined.

Examples of phosphate sources include phosphates such as potassium dihydrogen phosphate and dipotassium hydrogen phosphate, and phosphoric acid polymers such as pyrophosphoric acid. As the phosphate source, one kind of phosphate source may be used, or two or more kinds of phosphate source may be combined.

Examples of sulfur sources include inorganic sulfur compounds such as sulfates, thiosulfates, and sulfites, and sulfur-containing amino acids such as cysteine, cystine, and glutathione. As the sulfur source, one kind of sulfur source may be used, or two or more kinds of sulfur sources may be combined.

Specific examples of other various organic components and inorganic components include inorganic salts such as sodium chloride and potassium chloride; trace metals such as iron, manganese, magnesium, and calcium; vitamins such as vitamin B1, vitamin B2, vitamin B6, nicotinic acid, nicotinamide, and vitamin B12; amino acids; nucleic acids; organic components containing these, such as peptone, casamino acid, yeast extract, and soy protein decomposed products. As the other various organic components and inorganic components, one kind of component may be used, or two or more kinds of component may be used in combination.

In addition, in a case where an auxotrophic mutant that requires an amino acid or the like for growth is used, it is preferable to supplement a nutrient required for a medium. Furthermore, when a gene is introduced using a vector carrying an antibiotic resistance gene, it is preferable to add a corresponding antibiotic to a medium.

Culture conditions are not particularly limited as long as the bacterium of the present invention can grow and heparosan is produced and accumulated. Culture can be performed, for example, under normal conditions used for culturing bacteria. Culture conditions may be appropriately set by those skilled in the art.

The culture can be performed aerobically, for example, by aeration culture or shaking culture using a liquid medium. A culture temperature may be, for example, 30° C. to 37° C. A culture period may be, for example, 16 to 72 hours. The culture can be performed by batch culture, fed-batch culture, continuous culture, or a combination thereof. In addition, the culture may be divided into preculture and main culture. The preculture may be performed using, for example, a plate medium or a liquid medium.

By culturing the bacterium of the present invention as described above, heparosan accumulates in a medium.

A method for collecting heparosan from a culture solution is not particularly limited as long as heparosan can be collected. Examples of methods for collecting heparosan from a culture solution include methods described in Examples. Specifically, for example, a culture supernatant can be separated from a culture solution, and next, heparosan in the supernatant can be precipitated by ethanol precipitation. An amount of ethanol added may be, for example, in 2.5 to 3.5 times of an amount of a supernatant liquid. For the precipitation of heparosan, not only ethanol but also an organic solvent optionally miscible with water can be used.

Examples of organic solvents include ethanol, methanol, n-propanol, isopropanol, n-butanol, t-butanol, sec-butanol, propylene glycol, acetonitrile, acetone, DMF, DMSO, N-methylpyrrolidone, pyridine, 1,2-dimethoxyethane, 1,4-dioxane, and THF.

Further examples of the method for collecting heparosan from a culture solution include a method comprising purifying heparosan by targeting Kdo which is present at the terminal of heparosan.

The precipitated heparosan can be dissolved, for example, in water that is twice an amount of an original supernatant. The collected heparosan may contain components such as bacterial cells, medium components, water, and metabolic by-products of bacteria, in addition to heparosan. Heparosan may be purified to a desired degree. A degree of purity of heparosan may be, for example, 30% (w/w) or more, 50% (w/w) or more, 70% (w/w) or more, 80% (w/w) or more, 90% (w/w) or more, or 95% (w/w) or more.

Detection and quantification of heparosan can be performed by a known method. Specifically, for example, heparosan can be detected and quantified by a carbazole method as will be described later in Examples. The carbazole method is a method widely used as a method for quantifying uronic acid, and it is possible to detect and quantify heparosan by thermally reacting heparosan with carbazole in the presence of sulfuric acid, and measuring absorption at 530 nm by the generated coloring substance [Bitter T. and Muir H.M., (1962) “A modified uronic acid carbazole reaction.” Analytical Biochemistry, 4 (4):330-334]. In addition, for example, heparosan can be detected and quantified by treating heparosan with heparinase III, which is a heparosan-degrading enzyme, and performing disaccharide composition analysis.

### EXAMPLES

Examples are shown below, but the present invention is not limited to the following examples.

### Example 1

### Construction of Gene-deleted Strain

### (A) Construction of Marker Gene Fragment for Gene Deletion

A DNA fragment containing a chloramphenicol resistance gene (cat) and a levansucrase gene (sacB) used as marker genes for deletion of genes of Escherichia coli using homologous recombination was prepared as follows.

PCR was performed using synthetic DNAs consisting of the nucleotide sequences shown in SEQ ID NOs: 1 and 2 as a primer set and using a plasmid pHSG396 (Takara Bio Inc.) as a template, and thereby a DNA fragment comprising the cat gene was obtained. The PCR was performed using a PrimeSTAR Max DNA polymerase (Takara Bio Inc.) according to the description in the instruction manual. In addition, PCR was performed using synthetic DNAs consisting of the nucleotide sequences shown in SEQ ID NOs: 3 and 4 as a primer set and using pMOB3 (derived from ATCC 77282) as a template, and thereby a DNA fragment comprising the sacB gene was obtained.

After purifying the DNA fragment comprising the cat gene and the DNA fragment comprising the sacB gene, the DNA fragments were cut with Sail. Phenol/chloroform treatment and ethanol precipitation were performed, and the two fragments were mixed at an equimolar ratio and ligated using a DNA ligation Kit Ver.2 (Takara Bio Inc.). The ligation reaction solution was subjected to phenol/chloroform treatment and purified by ethanol precipitation, and PCR was performed using thus obtained product as a template and using synthetic DNAs consisting of the nucleotide sequences shown in SEQ ID NOs: 5 and 6 as a primer set. The resulting amplified DNA was purified using a Qiaquick PCR purification kit (Qiagen), and thereby a DNA fragment (a cat-sacB fragment) comprising the cat gene and the sacB gene was obtained.

### (B) Construction of YhbJ -Deficient Strain

The first PCR was performed using synthetic DNAs consisting of the nucleotide sequences shown in SEQ ID NOs: 7 and 8, and synthetic DNAs consisting of nucleotide sequences shown in SEQ ID NOs: 9 and 10 as primer sets, and using a genomic DNA of an Escherichia coli Nissle 1917 strain [DSM 6601, Mutaflor (Pharma-Zentrale GmbH), hereinafter abbreviated as a Nissle strain] prepared by a conventional method as a template. As a result, DNA fragments respectively comprising 1000 bp upstream from the vicinity of the start codon of the yhbJ gene and about 1000 bp downstream from the vicinity of the stop codon of the yhbJ gene were obtained. The PCR was performed using a PrimeSTAR Max DNA Polymerase (Takara Bio Inc.) according to the description in the instruction manual.

The second PCR was performed using, as a template, a mixture obtained by mixing the amplified product purified by using a QIAquick PCR Purification Kit (manufactured by Qiagen) and the cat-sacB fragment at an equimolar ratio. The PCR was performed using a PrimeSTAR GXL DNA Polymerase (Takara Bio Inc.) according to the description in the instruction manual. For the primer set, synthetic DNAs consisting of the nucleotide sequences shown in SEQ ID NOs: 7 and 10 was used. The amplified product was subjected to agarose gel electrophoresis to separate about 4.6 kbp of a DNA fragment, and thereby a DNA fragment comprising a yhbJ peripheral region into which the cat-sacB fragment had been inserted was obtained.

In addition, the first PCR was performed using synthetic DNAs consisting of the nucleotide sequences shown in SEQ ID NOs: 7 and 11, and synthetic DNAs consisting of the nucleotide sequences shown in SEQ ID NOs: 12 and 10 as primer sets, and using a genomic DNA of an Escherichia coli Nissle strain as a template. As a result, DNA fragments respectively comprising 1000 bp upstream from the vicinity of the start codon of the yhbJ gene and about 1000 bp downstream from the vicinity of the stop codon of the yhbJ gene were obtained.

The second PCR was performed using a mixture obtained by mixing the purified amplified product at an equimolar ratio as a template. For the primer set, a synthetic DNA consisting of nucleotide sequences shown in 7 and 10 was used. The amplified product was subjected to agarose gel electrophoresis to separate about 2.0 kbp of a DNA fragment, and thereby a DNA fragment comprising a yhbJ peripheral region deficient of the yhbJ gene was obtained.

Next, the Escherichia coli Nissle strain (a Nissle/pKD46 strain) retaining pKD46 was cultured in an LB medium [10 g/L bactotripton (manufactured by Difco), 5 g/L yeast extract (manufactured by Difco), and 5 g/L sodium chloride] in the presence of 15 g/L of L-arabinose and 100 mg/L of ampicillin. A plasmid pKD46 has a lambda Red recombinase gene, and therefore, expression of the gene can be induced by L-arabinose. Accordingly, when Escherichia coli retaining pKD46 which was grown in the presence of L-arabinose is transformed using a linear DNA, homologous recombination occurs at a high frequency. In addition, because pKD46 has a temperature-sensitive replication origin, the plasmid can be easily dropped off by growing pKD46 at 42° C. Competent cells of the Nissle/pKD46 strain were prepared, and the DNA fragment comprising the yhbJ peripheral region into which the cat-sacB fragment obtained above was inserted was introduced thereto by electroporation.

The obtained transformant was applied to an LB agar medium containing 15 mg/L of chloramphenicol and 100 mg/L of ampicillin (LB + chloramphenicol + ampicillin), and was cultured, and chloramphenicol resistance colonies were selected. Because the strain in which the homologous recombination has occurred is resistant to chloramphenicol and sensitive to sucrose, the selected colonies were replicated in an LB agar medium containing 10% sucrose and 100 mg/L of ampicillin, and LB + chloramphenicol + ampicillin (LB + sucrose + ampicillin), and thereby a strain showing chloramphenicol resistance and sucrose sensitivity was selected.

Colony PCR was performed on the selected strain using synthetic DNAs consisting of the nucleotide sequences shown in SEQ ID NOs: 13 and 10 as a primer set, and it was checked that the cat-sacB fragment was inserted at the position of the yhbJ gene. The strain in which the cat-sacB fragment was inserted at the position of the yhbJ gene was cultured in the same manner as described above to prepare competent cells, and the DNA fragment which was obtained above and contains the yhbJ peripheral region deficient of the yhbJ gene was introduced thereto by electroporation.

The obtained transformant was cultured on a LB + sucrose agar medium, and sucrose resistance colonies were selected. Since the strain that underwent homologous recombination does not contain the cat-sacB fragment and is therefore sensitive to chloramphenicol and resistant to sucrose, the selected colonies were replicated in an LB + chloramphenicol agar medium and an LB + sucrose agar medium, and thereby a strain showing chloramphenicol sensitivity and sucrose resistance was selected.

Colony PCR was performed on the selected strain using synthetic DNAs consisting of the nucleotide sequences shown in SEQ ID NOs: 13 and 10 as a primer set, and it was checked that the yhbJ gene was deleted. A strain in which the yhbJ gene was checked to be deleted was applied to an LB agar medium and cultured at 42° C., and then a strain showing ampicillin sensitivity, that is, a strain from which pKD46 had been dropped off, was selected.

A strain in which the yhbJ gene was deficient was obtained as described above and was designated as an Escherichia coli NY strain.

### Example 2

### Construction of Gene-Enhanced Strain

A promoter-region-substituted strain of a kfiA gene was constructed by the following method. The first PCR was performed using synthetic DNAs consisting of the nucleotide sequences shown in SEQ ID NOs: 14 and 15, and synthetic DNAs consisting of the nucleotide sequences shown in SEQ ID NOs: 16 and 17 as primer sets, and using a genomic DNA of an Escherichia coli Nissle strain prepared by a general method as a template. As a result, DNA fragments respectively comprising 1000 bp upstream from around 100 bp upstream of the start codon of the kfiA gene and about 1000 bp downstream from around the start codon of the kfiA gene were obtained.

The second PCR was performed using, as a template, a mixture obtained by mixing the amplified product purified by using a QIAquick PCR Purification Kit (manufactured by Qiagen) and the cat-sacB fragment at an equimolar ratio. For the primer set, synthetic DNAs consisting of the nucleotide sequences shown in SEQ ID NOs: 14 and 17 was used. The amplified product was subjected to agarose gel electrophoresis to separate about 4.6 kbp of a DNA fragment, and thereby a DNA fragment comprising a promoter peripheral region of the kfiA gene into which the cat-sacB fragment had been inserted was obtained.

The first PCR was performed using synthetic DNAs consisting of the nucleotide sequences shown in SEQ ID NOs: 14 and 18, and synthetic DNAs consisting of the nucleotide sequences shown in SEQ ID NOs: 19 and 17 as primer sets, and using a genomic DNA of an Escherichia coli Nissle strain prepared by a general method as a template. As a result, DNA fragments respectively comprising 1000 bp upstream from around 100 bp upstream of the start codon of the kfiA gene and about 1000 bp downstream from around the start codon of the kfiA gene were obtained.

In addition, PCR was performed using synthetic DNAs consisting of the nucleotide sequences shown in SEQ ID NOs: 20 and 21 as a primer set and using a genomic DNA of an Escherichia coli W strain (ATCC 9637) prepared by a general method as a template, and thereby about 300 bp of a DNA fragment comprising a uspA promoter was obtained.

The second PCR was performed using a mixture obtained by mixing the purified amplified product at an equimolar ratio as a template. For the primer set, synthetic DNAs consisting of the nucleotide sequences shown in 14 and 17 was used. The amplified product was subjected to agarose gel electrophoresis to separate about 2.3 kbp of a DNA fragment, and thereby a DNA fragment deficient of the kfiA promoter region from the kfiA promoter peripheral region, but comprising the uspA promoter instead was obtained.

Next, the Escherichia coli Nissle strain (a Nissle/pKD46 strain) retaining the plasmid pKD46 containing a gene encoding gamma Red recombinase, and the Escherichia coli NY strain (a NY/pKD46 strain) constructed in Example 1 were cultured in the presence of 15 g/L of L-arabinose and 100 mg/L of ampicillin. Competent cells of both strains were prepared, and the DNA fragment comprising the kfiA promoter peripheral region into which the cat-sacB fragment obtained above was inserted was introduced thereto by electroporation.

The obtained transformant was cultured on an LB + chloramphenicol + ampicillin agar medium, and chloramphenicol-resistant colonies were selected. A strain showing chloramphenicol resistance and sucrose sensitivity was further selected from the selected colonies.

Colony PCR was performed on the selected strain using synthetic DNAs consisting of the nucleotide sequences shown in SEQ ID NOs: 22 and 17 as a primer set, and it was checked that the cat-sacB fragment was inserted into the kfiA promoter region.

The strain in which the cat-sacB fragment was inserted into the kfiA promoter region was cultured in the same manner as described above to prepare competent cells, and the DNA fragment which was obtained above, and which is deficient of the kfiA promoter region from the kfiA promoter peripheral region, but comprises the uspA promoter instead was introduced thereto by electroporation.

The obtained transformant was cultured on a LB + sucrose agar medium, and sucrose resistance colonies were selected. A strain showing chloramphenicol sensitivity and sucrose resistance was further selected from the selected colonies.

Colony PCR was performed on the selected strain using synthetic DNAs consisting of the nucleotide sequences shown in SEQ ID NOs: 22 and 17 as a primer set, and it was checked that the uspA promoter was inserted into the kfiA promoter region. A strain in which the uspA promoter was checked to be inserted into the kfiA promoter region was applied to an LB agar medium and cultured at 42° C., and then a strain showing ampicillin sensitivity, that is, a strain from which pKD46 had been dropped off, was selected.

Strains in which the uspA promoter was inserted into the kfiA promoter region as described above were obtained, and were respectively designated as an NA strain and an NYA strain of Escherichia coli.

### Example 3

### Heparosan Production Test Using NY Strain, NA Strain, and NYA Strain

### (A) Culture of Heparosan-Producing Strain

The yhbJ-deficient mutant NY strain obtained in Example 1, the kfiA-promoter-substituted strain NA strain and the kfiA-promoter-substituted yhbJ-deficient strain NYA strain which were obtained in Example 2, and a Nissle strain which is a parent strain were cultured at 30° C. for 24 hours on an LB agar medium, and were respectively inoculated into a 2 L baffle containing 330 mL of a preculture medium [10 g/L soy peptide (Hinute AM; manufactured by Fuji Oil Co., Ltd.), 5 g/L sodium chloride, 5 g/L yeast extract powder (AY-80; manufactured by Asahi Food and Healthcare Co., Ltd.) were adjusted with sodium hydroxide so that a pH became pH 7.2], and cultured at 30° C. for 18 hours.

Into a jar fermenter containing 760 mL of the main culture medium [in which 20 g/L glucose, 13.5 g/L potassium dihydrogen phosphate, 4 g/L diammonium phosphate, 1.7 g/L citric acid, 1.7 g/L magnesium sulfate heptahydrate, 10 mg/L thiamine hydrochloride, and 10 mL/L trace mineral solution were adjusted with 5 mol/L of sodium hydroxide so that a pH became pH 6.7, and glucose and magnesium sulfate heptahydrate were added separately after autoclave sterilization (at 120° C. for 20 minutes)], 40 mL of the obtained preculture was inoculated and cultured at 37° C. for 72 hours at a stirring speed of 800 rpm, and an aeration rate of 1.5 L/min.

The trace mineral solution refers to a solution in which 10 g/L iron sulfate heptahydrate, 2 g/L calcium chloride, 2.2 g/L zinc sulfate heptahydrate, 0.5 g/L manganese sulfate tetrahydrate, 1 g/L copper sulfate pentahydrate, 0.1 g/L hexaammonium heptamolybdate tetrahydrate, and 0.02 g/L sodium tetraborate decahydrate was dissolved in 5 mol/L hydrochloric acid.

From a timing when a glucose concentration in the culture solution became 0 g/L (0 hour) to 72 hours, a feed solution [500 g/L glucose, 33.6 g/L potassium dihydrogen phosphate, 14.3 g/L magnesium sulfate, 0.4 g/L thiamine hydrochloride, and 14.3 mL/L trace mineral solution] was added at 7.0 mL/hour. An amount of the feed solution added by the end of the culture was about 450 mL.

### (B) Partial Purification of Heparosan From Culture Solution of Heparosan-Producing Strain

After incubating the culture solution diluted 10 times with distilled water at 100° C. for 30 minutes, the bacterial cells were removed from the culture solution by centrifugation, and 200 microliter of the obtained supernatant was transferred to a 1.5 mL Eppendorf tube. After adding and mixing 40 microliter of a 0.5 mol/L aqueous solution of sodium sulfate, 400 microliter of a 10 g/L aqueous solution of hexadecylpyridinium chloride was added and mixed by inversion, and the mixture was allowed to stand at 37° C. for 1 hour.

The solution was centrifuged to form a precipitate, and the supernatant was removed. After washing the precipitate with distilled water, 100 microliter of a solution [an aqueous solution containing 0.5 mol/L sodium chloride and 4% (v/v) ethanol] was added to dissolve the precipitate. After further allowing to stand at 4° C. overnight, 900 microliter of a 0.25 mol/L aqueous sodium chloride solution was added and mixed, and thereby a crude heparosan solution was obtained. A blank was also prepared by treating 200 microliter of the main culture medium in the same manner.

### (C) Measurement of Amount of Heparosan Accumulated by Carbazole-Sulfuric Acid Method

While 20 microliter of the crude heparosan solution was ice-cooled, 100 microliter of a sulfuric acid solution [a solution in which 9.5 g/L sodium tetraborate decahydrate was dissolved in concentrated sulfuric acid] was added and mixed, followed by incubation at 100° C. for 10 minutes. While the solution was ice-cooled again, 4 microliter of a carbazole solution [a solution in which 1.25 g/L carbazole was dissolved in 100% ethanol] was added thereto and mixed, and incubated at 100° C. for 15 minutes.

After ice-cooling the solution again, a temperature was returned to room temperature, and an absorbance at 530 nm was measured using a microplate reader. Using 0, 0.1, and 0.2 g/L sodium glucuronate monohydrate for calibration curves, calibration curves were created with a horizontal axis as a glucuronic acid concentration (g/L), and a vertical axis as an absorbance at 530 nm.

A heparosan concentration was calculated according to the calculation expression described below. Where, a desired heparosan concentration represents H (g/L), the obtained calibration curve represents y = ax + b, the A530 measurement value of the crude heparosan sample represents h, the blank A530 measurement value represents k, and the final dilution ratio represents n. 0.5387 indicates a content of glucuronic acid in heparosan, and 216/234 indicates a content of sodium glucuronate in sodium glucuronate monohydrate.

$$\text{H} \left(\text{g} / \text{L}\right) = \frac{\left(\text{h} - \text{k} - \text{b}\right) \times 216 \times \text{n}}{\text{a} \times \text{0} \text{.5387} \times \text{234}}$$

### (D) Results of Measuring Amount of Heparosan Accumulated

Table 1 shows an amount of heparosan accumulated when it is measured by the above method.

TABLE 1

| Strain | Amount of heparosan accumulated (g/L) |
|---|---|
| Nissle | 3.9 |
| NY | 9.9 |
| NA | 5.5 |
| NYA | 12.5 |

As shown in Table 1, an amount of heparosan accumulated in the NY strain deficient of yhbJ was more than twice as high as that in the parent strain Nissle. In addition, even in the NA strain in which the kfiA promoter was substituted by the uspA promoter, an amount of heparosan accumulated was increased as compared with that in the parent strain Nissle. The NYA strain in which these two mutations were combined showed even higher heparosan productivity.

### Example 4

### Construction of Plasmid Expressing Gene Involved in Heparosan Production

PCR reaction was performed using the chromosomal DNA of the Escherichia coli Nissle strain as a template, and using synthetic DNAs consisting of the nucleotide sequences shown in SEQ ID NOs: 23 and 24 as a primer set, and thereby about 3.3 kbp of a DNA fragment containing kpsC and kpsS gene regions (hereinafter referred to as kpsCS gene amplified fragment) was obtained.

A PCR reaction was performed using the pMW 118 vector as a template and using synthetic DNAs consisting of the nucleotide sequences shown in SEQ ID NOs: 25 and 26 as a primer set, and thereby about 4 kbp of a pMW 118 linear DNA fragment was obtained. In addition, a PCR reaction was performed using a chromosomal DNA of an Escherichia coli W strain (ATCC 9637) as a template, and using synthetic DNAs consisting of the nucleotide sequences shown in SEQ ID NOs: 20 and 21 as a primer set, and thereby about 300 bp of a DNA fragment comprising the uspA promoter region was obtained.

The pMW 118 linear DNA fragment, the DNA fragment containing the uspA promoter region, and the kpsCS gene amplified fragment obtained as above were mixed and ligated using an In-Fusion HD Cloning Kit (Takara Bio Inc.).

An Escherichia coli DH5 alpha strain was transformed using the obtained ligated DNA, and transformants were selected using ampicillin resistance as an index. A plasmid was extracted from the transformant according to a known method, and a PCR reaction was performed using synthetic DNAs consisting of the nucleotide sequences shown in SEQ ID NOs: 27 and 24 as a primer set, and thereby it was checked that the gene expression plasmid was obtained, which was designated as pMW118-kpsCS.

In addition, a PCR reaction was performed using a chromosomal DNA of the Escherichia coli Nissle strain as a template, and using synthetic DNAs consisting of the nucleotide sequences shown in SEQ ID NOs: 28 and 24, and synthetic DNAs consisting of the nucleotide sequences shown in SEQ ID NOs: 29 and 24 as primer sets, and thereby about 1.2 kbp of a DNA fragment containing the kpsS gene region (hereinafter referred to as a kpsS gene amplified fragment) and about 7.9 kbp of a DNA fragment containing the kpsF, kpsE, kpsD, kpsU, kpsC, and kpsS gene regions (hereinafter referred to as a kpsFEDUCS gene amplified fragment) were respectively obtained.

Furthermore, a PCR reaction was performed using the plasmid pMW118-kpsCS obtained above as a template, and using synthetic DNAs consisting of the nucleotide sequences shown in SEQ ID NOs: 25 and 21 as a primer set, and thereby about 4.3 kbp of a pMW118 linear DNA fragment containing the uspA promoter sequence was obtained.

The pMW118 linear DNA fragment containing the uspA promoter sequence, and the kpsS gene amplified fragment, which were obtained above, were mixed and ligated using an In-Fusion HD Cloning Kit (Takara Bio Inc.). In the same manner, the pMW118 linear DNA fragment comprising the uspA promoter sequence, and the kpsFEDUCS gene amplified fragment were mixed and ligated.

An Escherichia coli DH5 alpha strain was transformed using each of the ligated DNAs obtained, and transformants were selected using ampicillin resistance as an index. A plasmid was extracted from the transformant according to a known method, and a PCR reaction was performed using synthetic DNAs consisting of the nucleotide sequences shown in SEQ ID NOs: 20 and 24 as a primer set for the plasmid to which the kpsS gene amplified fragment was ligated, and using synthetic DNAs consisting of the nucleotide sequences shown in SEQ ID NOs: 27 and 30 and synthetic DNAs consisting of the nucleotide sequences shown in SEQ ID NOs: 31 and 32 as a primer set for the plasmid to which the kpsFEDUCS gene amplified fragment was ligated. Accordingly, it was checked that plasmids expressing the genes were respectively obtained, which were respectively designated as pMW118-kpsS and pMW118-kpsFEDUCS.

### Example 5

### Heparosan Production Test Using Strain Retaining Gene Expression Plasmid - 1

The pMW 1 18 plasmid, and pMW 118-PuspA-kpsS, pMW 118-PuspA-kpsCS, and pMW118-PuspA-kpsFEDUCS, which were obtained in Example 4, were respectively transformed to the NY strain obtained in Example 1. The obtained transformants were respectively designated as an NY/pMW118 strain, an NY/pMW118-PuspA-kpsS strain, an NY/pMW118-PuspA-kpsCS strain, and an NY/ pMW118-PuspA-kpsFEDUCS strain.

The transformants obtained above were inoculated into a large test tube containing 5 mL of an LB medium containing 100 mg/L of ampicillin, and cultured at 37° C. for 15 hours. The 1% culture solution was inoculated to a test tube containing 5 mL of an R medium containing 100 mg/L of ampicillin [in which 20 g/L glucose, 13.5 g/L potassium dihydrogen phosphate, 4 g/L diammonium phosphate, 1.7 g/L citric acid, 1 g/L magnesium sulfate heptahydrate, 10 mg/L thiamine hydrochloride, and 10 mL/L trace mineral solution were adjusted with 5 mol/L of sodium hydroxide so that a pH became pH 6.8, and glucose and magnesium sulfate heptahydrate were added separately after autoclave sterilization (at 120° C. for 20 minutes)], and cultured at 37° C. for 24 hours. In addition, the same operation was performed for the NY strain under the condition that ampicillin was not contained.

The obtained culture solution was treated by the method described in Example 3, and an amount of heparosan accumulated in the culture solution was measured. The results are shown in Table 2.

TABLE 2

| Strain | Amount of heparosan accumulated (mg/L) |
|---|---|
| NY | 192 |
| NY/pMW118 | 220 |
| NY/pMW118-PuspA-kpsS | 349 |
| NY/pMW118-PuspA-kpsCS | 328 |
| NY/pMW118-PuspA-kpsFEDUCS | 348 |

As shown in Table 2, in the NY/pMW118-PuspA-kpsS strain expressing kpsS, an amount of heparosan accumulated was clearly increased as compared with that in the parent strain NY, and the NY/pMW118 strain retaining only the pMW118 vector.

Meanwhile, based on the result that the NY/pMW118-PuspA-kpsCS strain expressing kpsC and kpsS and the NY/pMW118-PuspA-kpsFEDUCS strain expressing kpsF, kpsE, kpsD, kpsU, kpsC, and kpsS showed almost the same heparosan productivity as the NY/pMW118-PuspA-kpsS strain, it was found that an effect of improving a heparosan-producing ability was sufficiently obtained by enhancing expression of only kpsS in kpsFEDUCS, which is a heparosan production gene group.

### Example 6

### Heparosan Production Test Using Strain Retaining Gene Expression Plasmid - 2

The pMW1 18 plasmid, and pMW 118-PuspA-kpsS, pMW 118-PuspA-kpsCS, and pMW118-PuspA-kpsFEDUCS, which were obtained in Example 4, were respectively transformed to a wild-type strain of Escherichia coli Nissle. The obtained transformants were respectively designated as an N/pMW118 strain, an N/ pMW118-PuspA-kpsS strain, an N/pMW118-PuspA-kpsCS strain, and an N/ pMW118-PuspA-kpsFEDUCS strain.

The transformants obtained above were inoculated into a large test tube containing 5 mL of an LB medium containing 100 mg/L of ampicillin, and cultured at 37° C. for 15 hours. The 1% culture solution was inoculated to a test tube containing 5 mL of an R medium containing 100 mg/L of ampicillin [in which 20 g/L glucose, 13.5 g/L potassium dihydrogen phosphate, 4 g/L diammonium phosphate, 1.7 g/L citric acid, 1 g/L magnesium sulfate heptahydrate, 10 mg/L thiamine hydrochloride, and 10 mL/L trace mineral solution were adjusted with 5 mol/L of sodium hydroxide so that a pH became pH 6.8, and glucose and magnesium sulfate heptahydrate were added separately after autoclave sterilization (at 120° C. for 20 minutes)], and cultured at 37° C. for 24 hours. In addition, the same operation was performed for the wild-type Nissle strain under the condition that ampicillin was not contained.

The obtained culture solution was treated by the method described in Example 3, and an amount of heparosan accumulated in the culture solution was measured. The results are shown in Table 3.

TABLE 3

| Strain | Amount of heparosan accumulated (Ratio when wild-type Nissle strain is 1.0) |
|---|---|
| Wild-type Nissle strain | 1.0 |
| N/pMW118 | 0.9 |
| N/pMW118-PuspA-kpsS | 3.3 |
| N/pMW118-PuspA-kpsCS | 1.7 |
| N/pMW118-PuspA-kpsFEDUCS | 1.8 |

As shown in Table 3, in the N/pMW 118-PuspA-kpsS strain expressing kpsS, an amount of heparosan accumulated was increased by three times or more as compared with that in the parent wild-type Nissle strain, and the N/pMW1 18 strain retaining only the pMW 118 vector.

Meanwhile, in the N/pMW 118-PuspA-kpsCS strain expressing kpsC and kpsS and the N/pMW118-PuspA-kpsFEDUCS strain expressing kpsF, kpsE, kpsD, kpsU, kpsC, and kpsS, titer was improved by at most 1.8 times of the parent strain. In this regard, it was found that an effect of improving a heparosan-producing ability was sufficiently obtained by enhancing expression of only kpsS, among the heparosan-producing gene group kpsFEDUCS, and that the heparosan-producing ability became higher when the expression of kpsC and kpsFEDU was not enhanced, rather than being enhanced.

While the invention has been described in detail and with reference to specific embodiments thereof, it will be apparent to one skill in the art that various changes and modifications can be made therein without departing from the spirit and scope thereof. All references cited herein are incorporated in their entirety. This application is based on International application No. PCT/JP2020/015384 filed on Apr. 3, 2020, the entire contents of which are incorporated hereinto by reference.

1. A method for producing heparosan, comprising:
culturing a bacterium of the genus Escherichia which has the following genetic modification (1) and has a heparosan-producing ability in a medium to produce heparosan in the medium:
(1) a genetic modification increasing an expression of a kpsS gene.

2. The method for producing heparosan according to claim 1, wherein the bacterium of the genus Escherichia further has at least one of the following genetic modifications (2) and (3):
(2) a genetic modification increasing an expression of at least one gene selected from a kfiA gene, a kfiB gene, a kfiC gene, and a kfiD gene, and
(3) a genetic modification causing loss of a function of a yhbJ gene.

3. The method for producing heparosan according to claim 1, wherein the genetic modification (1) is at least one of modifying an expression control region of the kpsS gene and increasing the copy number of the kpsS gene.

4. The method for producing heparosan according to claim 2, wherein the genetic modification (2) is at least one of modifying an expression control region of at least one gene selected from the kfiA gene, the kfiB gene, the kfiC gene, and the kfiD gene, and increasing the copy number of at least one gene selected from the kfiA gene, the kfiB gene, the kfiC gene, and the kfiD gene.

5. The method for producing heparosan according to claim 2, wherein the genetic modification (3) is deletion of the yhbJ gene.

6. The method for producing heparosan according to claim 1, wherein the bacterium of the genus Escherichia is Escherichia coli.

7. The method for producing heparosan according to claim 1, wherein the kpsS gene is a DNA comprising the nucleotide sequence shown in SEQ ID NO: 33, or a DNA comprising a nucleotide sequence that has 90% or more identity to the nucleotide sequence shown in SEQ ID NO: 33 and having a property of increasing a heparosan-producing ability of the bacterium of the genus Escherichia having the heparosan-producing ability when an expression level is increased in the bacterium.

8. The method for producing heparosan according to claim 2, wherein
the kfiA gene is a DNA comprising the nucleotide sequence shown in SEQ ID NO: 34, or a DNA comprising a nucleotide sequence that has 90% or more identity to the nucleotide sequence shown in SEQ ID NO: 34 and having a property of increasing a heparosan-producing ability of the bacterium of the genus Escherichia having the heparosan-producing ability when an expression level is increased in the bacterium,
the kfiB gene is a DNA comprising the nucleotide sequence shown in SEQ ID NO: 35, or a DNA comprising a nucleotide sequence that has 90% or more identity to the nucleotide sequence shown in SEQ ID NO: 35 and having a property of increasing a heparosan-producing ability of the bacterium of the genus Escherichia having the heparosan-producing ability when an expression level is increased in the bacterium,
the kfiC gene is a DNA comprising the nucleotide sequence shown in SEQ ID NO: 36, or a DNA comprising a nucleotide sequence that has 90% or more identity to the nucleotide sequence shown in SEQ ID NO: 36 and having a property of increasing a heparosan-producing ability of the bacterium of the genus Escherichia having the heparosan-producing ability when an expression level is increased in the bacterium, and
the kfiD gene is a DNA comprising the nucleotide sequence shown in SEQ ID NO: 37, or a DNA comprising a nucleotide sequence that has 90% or more identity to the nucleotide sequence shown in SEQ ID NO: 37 and having a property of increasing a heparosan-producing ability of the bacterium of the genus Escherichia having the heparosan-producing ability when an expression level is increased in the bacterium.

9. The method for producing heparosan according to claim 2, wherein the yhbJ gene is a DNA comprising the nucleotide sequence shown in SEQ ID NO: 38, or a DNA comprising a nucleotide sequence that has 90% or more identity to the nucleotide sequence shown in SEQ ID NO: 38 and having a property of increasing a heparosan-producing ability of the bacterium of the genus Escherichia having the heparosan-producing ability when an expression level is decreased in the bacterium.

10. The method for producing heparosan according to claim 1, wherein the bacterium of the genus Escherichia does not have the following genetic modification (4):
(4) a genetic modification increasing an expression of a kpsC gene.

11. A bacterium of the genus Escherichia which has a heparosan-producing ability and has the following genetic modification (1):
(1) a genetic modification increasing an expression of a kpsS gene.

12. The bacterium of the genus Escherichia according to claim 11, which further has at least one of the following genetic modifications (2) and (3):
(2) a genetic modification increasing an expression of at least one gene selected from a kfiA gene, a kfiB gene, a kfiC gene, and a kfiD gene, and
(3) a genetic modification causing loss of a function of a yhbJ gene.

13. The bacterium of the genus Escherichia according to claim 11, which does not have the following genetic modification (4):
(4) a genetic modification increasing an expression of a kpsC gene.
